(19) United States
(12) Patent Application Publication
(10) Pub. No.: US 2025/0019771 A1
(43) Pub. Date: Jan. 16, 2025

(54) TRANSCRIPTOMIC SIGNATURE BASED ON HERVs EXPRESSION TO CHARACTERIZE LEUKEMIC STEM CELLS AND USEFUL AS A LSC MARKER

(71) Applicants: ERVIMMUNE, LYON CEDEX 08 (FR); CENTRE LEON BERARD, LYON (FR); UNIVERSITE CLAUDE BERNARD LYON 1, Villeurbanne (FR); INSTITUT NATIONAL DE LA SANTE ET DE LA RECHERCHE MEDICALE (INSERM), Paris (FR); CENTRE NATIONAL DE LA RECHERCHE SCIENTIFIQUE, Paris (FR)

(72) Inventors: Stéphane DEPIL, LYON CEDEX 8 (FR); Vincent ALCAZER, PIERRE-BENITE (FR)

(73) Assignees: ERVIMMUNE, LYON CEDEX 08 (FR); CENTRE LEON BERARD, LYON (FR); UNIVERSITE CLAUDE BERNARD LYON 1, Villeurbanne (FR); INSTITUT NATIONAL DE LA SANTE ET DE LA RECHERCHE MEDICALE (INSERM), Paris (FR); CENTRE NATIONAL DE LA RECHERCHE SCIENTIFIQUE, Paris (FR)

(21) Appl. No.: 18/713,383

(22) PCT Filed: Nov. 25, 2022
(86) PCT No.: PCT/EP2022/083377

§ 371 Date: May 24, 2024

(30) Foreign Application Priority Data
Nov. 26, 2021 (EP) 21306648.3

(51) Int. Cl. C12Q 1/6886; C12Q 1/70

(52) U.S. Cl. CPC C12Q 1/6886; C12Q 1/702; C12Q 2600/106; C12Q 2600/118; C12Q 2600/158

(57) ABSTRACT

A transcriptomic signature based on Human endogenous retroviruses (HERVs) expression to characterize leukemic stem cells. In particular, determining the presence of Leukemic Stem Cells (LSCs) in a patient. In an aspect, determining the presence of, or quantifying, LSCs in a patient. Also, gene-related methods for the identification of high-risk acute myeloid leukemia (AML) patients, methods of predicting response to treatment, methods to evaluate (minimal) residual disease during follow-up, methods to determine relapse risk, and methods of treatment of patients following implementation of the former methods.

### FIELD

The present invention concerns the use of a transcriptomic signature based on Human endogenous retroviruses (HERVs) expression to characterize leukemic stem cells. In particular, the invention allows determining the presence of Leukemic Stem Cells (LSCs) in a patient. In an aspect, the invention allows determining the presence of, or quantifying, LSCs in a patient. The invention relates to gene-related methods for the identification of high-risk acute myeloid leukemia (AML) patients, methods of predicting response to treatment, methods to evaluate (minimal) residual disease during follow-up, methods to determine relapse risk, and methods of treatment of patients following implementation of the former methods.

### BACKGROUND

HERVs represent 8% of the human genome (1). These sequences are remnants of ancestral germline infections by exogenous retroviruses (2). The original sequence of a HERV is that of an exogenous retrovirus, with two promoter long-terminal repeat (LTR) sequences surrounding the virus open-reading frames (ORFs): gag, pro, pol and env (3). However, after millions of years of evolution, these ORFs have been deeply altered, and there is currently no description of any autonomous fully infectious HERV (4).

The long-standing belief is that HERVs are repressed by epigenetic mechanisms and are thus not expressed, or only poorly, in normal tissues (5). However, recent studies have shown that HERV expression can be detected in a vast range of normal tissues (6). Different pathological conditions can lead to aberrant HERV expression, as it has now been largely described in auto-immune diseases (4) and in cancers (7), where HERVs have been the subject of many studies over the last years. Indeed, it was reported that HERVs could participate in oncogenesis by inducing chromosomal instability, promoting aberrant gene expression with their LTR or by impacting the immune system with their RNA and protein products (7). HERVs could thus play a prominent role in cancer immunity, increasing tumor immunogenicity by promoting (i) an innate immune response triggered by the viral defense pathway induced by their nucleic acid intermediates, and (ii) an adaptive immune response by forming a pool of tumor-associated antigens (8).

Acute Myeloid Leukemia (AML) is a heterogeneous disease characterized by the clonal expansion of myeloid progenitor and stem cells (9). While some AML subtypes are characterized by recurrent genetic translocations or mutations associated with particular prognoses, most AMLs present a normal or complex karyotype, and identifying key factors that predict treatment resistance in these patients represents a major challenge (9,10). Aside from disease stratification, AML also belongs to malignancies with the lowest mutational burdens (11), and finding tumor-specific antigens for immunotherapeutic approaches remains very difficult as the frequency of mutations creating neoantigens is expected to be low. In this context, HERV-derived antigens could represent a unique source of non-conventional epitopes that could be exploited for the development of new immunotherapies (12).

The high rate of relapse in AML has been attributed to the persistence of leukaemia stem cells (LSCs), which possess a number of stem cell properties, such as quiescence, that are linked to therapy resistance. Stanley W K Ng et al. (18) develops predictive and prognostic biomarkers related to stemness. They generated a list of genes that are differentially expressed between 138 LSC+ and 89 LSC− cell fractions from 78 AML patients validated by xenotransplantation. The core transcriptional components of stemness relevant to clinical outcomes were extracted using sparse regression analysis of LSC gene expression against survival in a large training cohort, generating a 17-gene LSC score (LSC17). The so-called LSC17 score allows for prediction of initial therapy resistance. Patients with high LSC17 scores are considered having poor outcomes with current treatments including allogeneic stem cell transplantation.

To date, little is known about the expression of HERVs in AML and its relevance as either a biomarker or a therapeutic target. Evidence of HERV-K/HML-2 expression in AML cells was shown as early as 1993 and confirmed in the early 2000s (13,14). Few studies then focused on HERVs in AML until the late 2010s, with the demonstration that azacytidine (Aza) activates the transcription of different HERVs, potentially contributing to its clinical effects (15). The exact role of HERVs in Aza therapy is however a matter of debate, with recent evidence arguing in favor of a HERV-independent therapeutic effect (16). More recently, a link was established between HERVs and the expression of surrounding genes in AML, suggesting a regulatory role of these retroelements (17). Albeit, few data exist on HERV expression and their immune impact in AML, with studies relying on non-exhaustive quantification methods, such as polymerase chain reaction (PCR), or focusing only on a few HERV loci.

### SUMMARY

Different signatures have been established based on HERV expression from several RNA sequencing data to characterize Leukemic Cell Cells (LSC). The concrete use of HERVs to characterize cell populations in AML is rendered possible for the first time.

To establish the HERV-LSC signature or Leukemic Cell Score, correlations between the expression of each unique HERV and the validated LSC17 score (18) were computed independently in 4 public bulk RNA-seq datasets (AMLCG, TCGA, BEAT and LEUCEGENE). 47 HERVs showing a significant correlation (False Discovery rate (FDR)-adjusted p-value <0.05) with the LSC17 score in at least 2 datasets and with concordant results (i.e. correlated in the same direction in each dataset) were retained to build the final signature. The HERV-LSC signature was then calculated as the mean 47-HERV expression pondered by the mean correlation coefficient of each HERV with the LSC17 score. This signature was then validated in the sorted cells from Corces et al. (19) with a classification approach, for LSC alone or LSC and Blasts. Receiver Operating Characteristic (ROC) curves and Area Under the Curve (AUC) were drawn and calculated with the plotROC R package. The final signature is provided in table 1. This signature separates LSCs from all other cells with an AUC of 0.75 (FIG. 1). Misclassified cells are mainly attributed to blasts, as confirmed by the very good AUC of 0.92 when regrouping LSCs and blasts.

Using the unique information provided by the HERV retrotranscriptome, it has thus been possible to build a signature based on 47 HERVs that allowed a robust classification of stem cells among (1) normal bone-marrow cells and (2) leukemic bone-marrow cells. Misclassified cells are mostly represented by blast cells. Subset of these 47 signatures may also be used, but the use of such a subset may lead to a sensitivity and/or specificity loss. The invention thus encompasses use of such a subset, especially as defined infra.

The HERV-LSC signature represents an original and powerful tool to determine the presence of Leukemic Stem Cells (LSCs), to evaluate AML prognosis, as a marker of remaining LSCs, which remaining LSCs could be responsible for relapses of AML, or to evaluate minimal residual disease during follow-up.

The present invention thus concerns a method of determining an HERV-LSC signature or score. This method may be used to identify high-risk acute myeloid leukemia (AML) patients, to predict response to AML treatment, to evaluate minimal or residual disease during follow-up, to determine relapse risk in an AML patient that is being treated or has been treated, and all these methods may be used to decide treating the patient against AML, especially with adapted treatment protocol and/or more or less intensive AML therapy. The method may thus be used to prognose or classify a subject in AML patient or in risk of developing AML or having an AML relapse.

The method comprises determining from a subject's sample, expression of HERVs selected from those 47 listed in Table 1, preferably those having an absolute coefficient >0.4, this coefficient being indicated in Table 1. The method also comprises determining the expression value of the selected HERVs in the subject's sample. Then the method comprises the calculation of a score as follows: one multiply each HERVs' expression value by its coefficient provided in Table 1, which gives a pondered HERVs' expression for each one of those HERVs, then the score is obtained by calculating the mean of each one of the pondered HERVs' expressions.

In an aspect, there is provided a method of determining an HERV-LSC signature or score. More particularly, the method aims at prognosing or classifying a subject with AML, or in risk of developing AML or having an AML relapse, comprising determining this score in a subject's sample. The score is specific and predictive to an AML status. A high score is of bad prognosis. The score may be used to monitor the efficacy of an anticancer therapy, a decrease of the score after and/or during therapy being a sign of at least some efficacy of said therapy. However, the method allows the determination of the score after therapy in order to know the presence or the number of remaining LSCs in said patient. This allows prognosis of AML relapse.

### BRIEF DESCRIPTION OF THE DRAWINGS

FIG. 1: ROC-curve of LSC and LSC-blast classification according to the LSC signature established on 47 different HERVs.

LSC: Leukemic Stem Cell, ROC: Receiver Operating Characteristic Curve, ssGSVA: Single Sample Genes-set Variation Analysis, WBC: White Blood Count.

### DETAILED DESCRIPTION

In particular, the score is assessed with the detection or expression of at least 10, at least 15, at least 20, at least 25, or the 29 of the 29-HERVs of Table 1 with an absolute coefficient >0.4, coefficient indicated in the same Table 1 (HERVs N° 1-15, and 34-47).

Preferably, the score is assessed with the whole 47-HERVs of said table 1 (HERVs N° 1-47), or with a subset of 30, 31, 32, 33, 34, 35, 36, 37, 38, 39, 40, 41, 42, 43, 44, 45 or 46, especially including the 29-HERVs of Table 1 with an absolute coefficient >0.4 in the same Table 1 (HERVs N° 1-15, and 34-47).

Table 1 gives the identification (Row_id) of each one of HERVs N° 1-47, together with their corresponding locus on the GRCH38 human genome (e.g. ERVLE_16p13.3b, corresponds to locus 13.3b on the short arm (p) of chromosome 16) and their coefficients in the final signature. Using the Row_id, the open-source tool Telescope made available online (Bendall Matthew L et al., (Sep. 30, 2019), PLOS Computational Biology. 2019; 15 (9): e1006453, Telescope: Characterization of the retrotranscriptome by accurate estimation of transposable element expression. (https://journals.plos.org/ploscompbiol/article/comments?id=10.1371/journal.pcbi.1006453, https://github.com/mlbendall/telescope) and the genomic reference file in the international General Feature Format 2612_47HERVs_GRCH38_genomic_ref.gtf (available online at: github.com/VincentAlcazer/hervs_ref/blob/main/2612_47HERVs_GRCH38_genomic_ref.gtf), the skilled person has access to the exact genomic coordinates of each of the 47 HERV sequences in the GRCH38 version of the human genome (Table 2), and consequently to their corresponding nucleotide sequences.

Thus, in an aspect, the present invention relates to a method of determining an HERV-LSC signature or score, preferably to prognose or classify a subject with AML or in risk of developing AML or having an AML relapse, comprising determining from a subject's sample, expression of at least 10, at least 15, at least 20, at least 25, or preferably the 29 of the 29-HERVs N° 1-15, and 34-47, determining the expression value of each such expressed HERV, calculating the score as follows:
for each expressed HERV, one multiply the HERVs' expression value by its coefficient provided in Table 1, giving a pondered HERVs' expression for each one of those HERVs,
then calculating the score as the mean of each one of the pondered HERVs' expression.

The method may include additional HERVs selected from HERVs N° 16-33. In particular, the method may comprise determining from a subject's sample, expression of the whole 47-HERVs N° 1-47, or with a subset of 30, 31, 32, 33, 34, 35, 36, 37, 38, 39, 40, 41, 42, 43, 44, 45 or 46, preferably comprising the 29-HERVs N° 1-15, and 34-47. Then the score is calculated as explained before while taking into account all the HERVs expressed in said subject's sample.

Subject's sample may be any cell sample from a patient, e.g. an AML patient or a patient in risk of AML. The cell sample may be a bonne marrow sample or a peripheral blood sample containing white blood cells. The cell sample is preferably a (bulk) bone marrow sample.

In the method HERVs RNAs are recovered, cDNA are produced from these RNAs.

In the method, the RNA from the sample is fragmented and the fragments are reverse transcribed into cDNA fragments, or the RNA is reverse transcribed to cDNA and then fragmented to get cDNA fragments, before conducting the following steps.

The size of the RNA fragments may vary in large proportion as known by the skilled person. Typically, RNA fragments may have a size of from 50 to 100 base pairs, e.g. about 75 base pairs.

In an aspect, said cDNA fragments are sequenced and aligned back to a pre-sequenced reference human genome or human genome reference (1). It is convenient using a sequence aligner. These alignments are tested for overlap with said HERVs' sequences, and the number of overlap reads mapped to a gene is registered for each HERVs' sequence giving its expression value.

In an aspect, High-throughput sequencing with RNA, commonly referred to as RNA-Seq, is used. This method involves reverse transcribing RNA into cDNA, sequencing the cDNA, then mapping sequenced fragments of cDNA on a pre-sequenced reference genome or human genome reference as mentioned. In RNA-Seq, the RNA is fragmented and then reverse transcribed to cDNA (or reverse transcribed then fragmented). These cDNA fragments are then sequenced, producing reads that are aligned back to a pre-sequenced reference genome or human genome reference. The number of reads mapped to a gene or cDNA quantifies the expression level thereof and of the original RNA.

Thus, in an aspect, the method further comprises performing RNA-Seq, which is a so-called next generation sequencing (NGS).

Sequencing can be either non-targeted (total or polyA RNA-seq) or targeted.

When HERVs quantification is performed from RNA-Seq data, the method comprises aligning raw-reads (in particular generated by the sequencer) to human genome reference. It is convenient using a sequence aligner, preferably a fast or ultrafast sequence aligner, such as Bowtie2 v2.2.1 (21) with conservative parameters: (e.g. recommended parameters with Bowtie 2: —no-unal —score-min L, 0, 1.6-k 100 —very-sensitive-local).

A relevant description of the method is described in reference (20), the whole content of which is incorporated herein by reference.

Quantifying HERVs expression is then made, the number of reads mapped to a gene or cDNA quantifies the expression level thereof and of the original RNA.

Preferably, this quantification is made using a computer implemented method or an adequate software. The open-source tool Telescope (20) is a suitable one. A relevant description of the method is described in the previously mentioned reference (20). Telescope is available at https://github.com/mlbendall/telescope.

Quantifying genes is also made with any suitable tool such as HTseq (HTSeq 0.12.3 (24)) or featurecount with default parameters to directly obtain raw-counts corresponding to canonic genes.

Normalizing expression data taking genes raw count into account may then be realized, e.g. using DESEQ2's normalization with variance stabilizing transformation (VST), counts per million (CPM, counts scaled by total number of reads) and/or transcripts per million (TPM, counts per length of transcript per million reads mapped) for example.

The HERV-LSC score is then calculated for said patient. First, one multiply each HERVs' expression value, preferably normalized expression value by its coefficient provided in Table 1, giving the pondered expression. Then one calculate the score as the mean of each pondered HERVs' expression, say the mean of all the HERVs considered for measurement.

The more this value, the more the amount of residual LSC. Thus, the method may allow assessing a prognosis based on this value. In a first embodiment, this may be done using a relative classification, i.e. considering the continuous value for a given cohort, and classifying patients between them, patients with the lowest value having the best predicted outcome, and patient with the highest value the worse predicted outcome. In a second embodiment, this may be done using an absolute classification based on the score's terciles on the training cohort, patients with a score of more than 0.5 having a poor predicted outcome, and patients with a score of less or equal than 0.15 having a good predicted outcome.

Thus, the method may allow assessing a prognosis based on this value, for example by comparison to reference values or known patient groups.

In an embodiment, the reference group is a group of patients of known prognosis, and one calculates their pondered HERVs expression value and their score or signature as reference. Using a reference group of patients with a given prognosis or a corresponding known reference value, it is possible to classify a patient especially in good prognosis, medium prognosis, or bad prognosis, for example.

In another embodiment, the reference is or comprise the patient itself subjected to prognosis, wherein the reference patient is before AML treatment and allows to follow treatment effect, or the reference patient is the same at the time or at the end of AML treatment and prognosis is to evaluate response to AML treatment, to evaluate minimal or residual disease during follow-up, to determine relapse in said AML patient, for example.

In an embodiment, the HERV-LSC signature represents a surrogate marker of remaining LSCs that could be responsible for relapses of AML, or to evaluate minimal residual disease during follow-up. The case may be determined using reference values or reference group of known status.

In another aspect, the invention relates to the use of an anticancer drug for treating a subject against AML, wherein the subject had been previously identified as having an high risk AML by use of the above method.

In an aspect, the invention relates to a method of treating a subject against AML, comprising treating the patient with a cancer therapy against AML, in particular an aggressive cancer therapy, wherein the subject had been previously identified as being in a high risk group for AML or in risk of developing AML or having an AML relapse, by use of this method of determining an HERV-LSC signature or score.

The method of determination allows to calculate said score for the patient. If the score is equal or above the median value of a given population, the patient is qualified as being in a high risk group. If the score is below the median value of a given population, the patient is qualified as being in a low risk group.

The aggressive therapy is preferably an identified chemotherapy or an alternative therapy through enrollment into a clinical trial for a novel therapy.

In an aspect, the invention relates to a method of selecting a therapy for a subject with respect to AML, comprising the steps:
(a) classifying the subject into a high risk group or a low risk group according to the method as disclosed herein; and
(b) selecting an aggressive therapy, preferably intensified chemotherapy or monoclonal antibody therapy, or an alternative therapy through enrollment into a clinical trial for a novel therapy, for the high risk group or a less aggressive therapy, preferably standard chemotherapy, for the low risk group.

Any registered therapy or experimental/clinical trial therapy may be selected for a patient identified by the method of the invention as requiring an anti-AML therapy. This may include standard chemotherapy for AML, such as the one including cytosine arabinoside (Ara-c) in conjunction with an anthracycline, such as daunorubicin or the nucleoside analogue fludarabine. Of course any future chemotherapy molecule or protocol could be used in the present invention.

This may also include antibody therapies. Monoclonal antibody therapy may include the use of the following antibodies:
CSL360/CSL362 (Talacotuzumab)
The murine anti-human CD123 mAb 7G3 has been modified into two versions: chimeric CSL360 and humanized CSL362 (talacotuzumab). CSL360 has the variable region of 7G3 and is fused with the backbone of a human IgG1 through genetic engineering. CSL362 is a second version of CSL360, it is Fc optimized to bind CD16A on NK cells with better affinity, as well as affinity matured to better bind to CD123.
Lintuzumab and B1 835858
Lintuzumab (SGN-33, HuM195) is an unconjugated anti-CD33 mAb which has been tested in several clinical trials for AML in combination with standard induction chemotherapy. Another unconjugated anti-CD33 mAb, BI 836858, is Fc optimized through engineering, leading to improved NK cell-mediated ADCC relative to native antibody Fc.
Daratumumab (Darzalex), Isatuximab
Daratumumab is a fully human IgG1 kappa mAb that targets CD38. Another anti-CD38 antibody, isatuximab, has also been tested in MM, NHL, and CLL patients.
Multivalent Antibody Therapies
Multivalent antibodies with, bi-, tri- and quadri-specific binding domains are engineered constructs which combine specificities of two or more antibodies into one molecular product that is designed to bind to both a TAA and an activating receptor on the effector cells, typically a T cell or natural-killer (NK) cell. There are several different structural variants of bispecific antibodies, which, in turn, can be utilized to target various combinations of effector and tumour targets antigens. The first FDA-approved dual-binding antibody was blinatumomab, a bispecific T-cell engager (BiTE) developed by Amgen with specificities for CD3 and CD19 for treatment of acute lymphoblastic leukemia (ALL). BiTE format antibodies (tandem di-scFv) are engineered products involving combining the VL and VH domains of a monoclonal antibody into a single chain fragment variable (scFv) specific to an activating receptor (e.g., CD3) and further linked to the scFv of an antibody specific to a target antigen (e.g., CD19). It can also be applied to engineered antibody fragments with different formats than the BiTE, such as DART and Duobody, also increasing valency, such as with tri-specific antibodies.
anti, which is a CD33×CD3 specific BiTE for treatment of AML, developed by Amgen.

Any monoclonal antibody therapy or protocol, as well as innovative forms such as Bispecific Tandem Fragment Variable Format (BiTE, scBsTaFv), Dual-Affinity Retargeting (DART), Bispecific scFv Immunofusion, Bispecific Tandem Diabodies, Chemically Conjugated Bispecific Antibodies, Bispecific Full-Length Antibodies (Duobody and Biclonics), BiKEs and TriKEs, Toxin-Conjugated Antibody Therapy for AML, ADCs such as gemtuzumab ozogamicin, Vadastuximab Talirine (SGN33A) and IMGN779.

For a Review to which the skilled person may refer, see Brent A. Williams et al., Antibody Therapies for Acute Myeloid Leukemia: Unconjugated, Toxin-Conjugated, Radio-Conjugated and Multivalent Formats, J Clin Med. 2019 August; 8 (8): 1261; Published online 2019 Aug. 20. doi: 10.3390/jcm8081261, which is incorporated herein by reference.

TABLE 1

| Row_id | n_study | Coefficient | HERV No |
|---|---|---|---|
| ERVLB4_2q32.3 | 2 | 0.507183832 | 1 |
| ERVLE_16p13.3b | 2 | 0.501681707 | 2 |
| ERVLB4_2q13c | 2 | 0.49389202 | 3 |
| HERVH48_8q21.2 | 2 | 0.480036865 | 4 |
| HERVH_8q22.1c | 2 | 0.473093207 | 5 |
| MER41_9p13.3a | 2 | 0.470892447 | 6 |
| HERVL_5q13.3 | 2 | 0.447099459 | 7 |
| ERVLB4_8q21.13c | 2 | 0.442684087 | 8 |
| ERVLE_2q14.3g | 2 | 0.43058301 | 9 |
| HML2_7q11.21 | 2 | 0.412450241 | 10 |
| HML3_5p15.33d | 3 | 0.40965002 | 11 |
| HERVH_19q12 | 2 | 0.405353476 | 12 |
| HERVL_Xq13.3b | 2 | 0.401545261 | 13 |
| HERVW_3p11.1 | 3 | 0.400651062 | 14 |
| MER4_Xq13.1 | 2 | 0.399010999 | 15 |
| HUERSP3_9p23 | 2 | 0.398573124 | 16 |
| LTR46_19q13.43 | 2 | 0.396332553 | 17 |
| HERVH_3q21.3d | 2 | 0.396163605 | 18 |
| HML3_5p15.33c | 3 | 0.388344962 | 19 |
| HERVH_3q11.1 | 2 | 0.381952847 | 20 |
| ERVLE_4q31.21c | 2 | 0.360797685 | 21 |
| MER4B_12q22 | 3 | 0.357196649 | 22 |
| MER61_4q31.21a | 2 | 0.356647016 | 23 |
| HARLEQUIN_Xq23b | 2 | 0.34665982 | 24 |
| HERVL74_10q23.33 | 2 | 0.335099728 | 25 |
| HARLEQUIN_1q44 | 4 | 0.216775731 | 26 |
| HERVH_10q24.31a | 4 | 0.202687897 | 27 |
| HML6_19q13.41e | 3 | 0.178940562 | 28 |
| HERVK11_4q13.2 | 2 | −0.337516037 | 29 |
| HERVL_13q33.3 | 2 | −0.352018746 | 30 |
| ERV316A3_13q33.3a | 2 | −0.38722975 | 31 |
| ERVL_13q13.1a | 2 | −0.397275973 | 32 |
| ERVLB4_13q13.1 | 2 | −0.40628408 | 33 |
| ERV316A3_13q33.3c | 2 | −0.410735388 | 34 |
| PRIMA41_13q13.1 | 2 | −0.417379745 | 35 |
| HML5_7q22.3 | 2 | −0.421228515 | 36 |
| ERV316A3_13q33.3b | 2 | −0.427950383 | 37 |
| ERVLE_13q33.3e | 2 | −0.433574701 | 38 |
| ERVLE_13q33.3f | 2 | −0.442882815 | 39 |
| HERVH_1q41e | 3 | −0.459234876 | 40 |
| MER41_1q44a | 3 | −0.481155319 | 41 |
| MER4B_12p13.31c | 2 | −0.487409032 | 42 |
| MER41_1q44b | 2 | −0.490636595 | 43 |
| ERV316A3_9q21.33 | 2 | −0.497367476 | 44 |
| ERVLB4_22q11.21a | 2 | −0.507363365 | 45 |
| MER4_11p15.4a | 2 | −0.532284578 | 46 |
| LTR57_5q31.1 | 2 | −0.539771648 | 47 |

TABLE 2

| | | Start | End |
|---|---|---|---|
| | | position on | position on |
| Row_id | Chromosome | chromosome | chromosome |
| HERVH_1q41e | chr1 | 221947781 | 221952102 |
| MER41_1q44a | chr1 | 244815534 | 244818297 |
| MER41_1q44b | chr1 | 244818602 | 244821738 |
| HARLEQUIN_1q44 | chr1 | 247898331 | 247905895 |
| ERVLB4_2q13c | chr2 | 110379003 | 110381368 |
| ERVLE_2q14.3g | chr2 | 123772626 | 123774535 |
| ERVLB4_2q32.3 | chr2 | 192042435 | 192046614 |
| HERVW_3p11.1 | chr3 | 87940794 | 87946379 |
| HERVH_3q11.1 | chr3 | 93938055 | 93944071 |
| HERVH_3q21.3d | chr3 | 128959818 | 128965400 |
| HERVK11_4q13.2 | chr4 | 69191808 | 69199234 |
| MER61_4q31.21a | chr4 | 142656025 | 142661274 |
| ERVLE_4q31.21c | chr4 | 142859332 | 142861588 |
| HML3_5p15.33c | chr5 | 1577862 | 1580973 |
| HML3_5p15.33d | chr5 | 1581038 | 1585387 |
| HERVL_5q13.3 | chr5 | 74785888 | 74792672 |
| LTR57_5q31.1 | chr5 | 132281240 | 132287803 |
| HML2_7q11.21 | chr7 | 66004684 | 66007803 |
| HML5_7q22.3 | chr7 | 107386034 | 107391346 |
| ERVLB4_8q21.13c | chr8 | 78937023 | 78941378 |
| HERVH48_8q21.2 | chr8 | 85273771 | 85275053 |
| HERVH_8q22.1c | chr8 | 97827675 | 97831909 |
| HUERSP3_9p23 | chr9 | 9997607 | 10001203 |
| MER41_9p13.3a | chr9 | 33703617 | 33708862 |
| ERV316A3_9q21.33 | chr9 | 85630235 | 85631584 |
| HERVL74_10q23.33 | chr10 | 94673087 | 94675530 |
| HERVH_10q24.31a | chr10 | 100380920 | 100388398 |
| MER4_11p15.4a | chr11 | 3110563 | 3118570 |
| MER4B_12p13.31c | chr12 | 9984964 | 9987795 |
| MER4B_12q22 | chr12 | 93545453 | 93546576 |
| ERVL_13q13.1a | chr13 | 31683396 | 31683990 |
| PRIMA41_13q13.1 | chr13 | 31715164 | 31717353 |
| ERVLB4_13q13.1 | chr13 | 31718551 | 31719819 |
| HERVL_13q33.3 | chr13 | 109218916 | 109225445 |
| ERVLE_13q33.3e | chr13 | 109315443 | 109316147 |
| ERVLE_13q33.3f | chr13 | 109323457 | 109325339 |
| ERV316A3_13q33.3a | chr13 | 109445952 | 109446780 |
| ERV316A3_13q33.3b | chr13 | 109465321 | 109466932 |
| ERV316A3_13q33.3c | chr13 | 109467032 | 109467884 |
| ERVLE_16p13.3b | chr16 | 3154472 | 3155580 |
| HERVH_19q12 | chr19 | 28431623 | 28435712 |
| HML6_19q13.41e | chr19 | 52984535 | 52989576 |
| LTR46_19q13.43 | chr19 | 57651316 | 57656645 |
| ERVLB4_22q11.21a | chr22 | 19787055 | 19787825 |
| MER4_Xq13.1 | chrX | 72696375 | 72703208 |
| HERVL_Xq13.3b | chrX | 75470018 | 75474643 |
| HARLEQUIN_Xq23b | chrX | 115916433 | 115924222 |

Table 2 summarizes the genomic coordinates (start position and end position on chromosome) of each of the 47 HERV sequences in the GRCH38 version of the human genome.

As an example, concerning the Row_id “ERVLB4_2q32.3”, the “2” value corresponds to chromosome 2 of the human genome, the letter (q) corresponds to the long arm of the corresponding chromosome (alternatively the letter (p) corresponds to the short arm of the corresponding chromosome) and 32.3 corresponds to the locus of the gene of the corresponding chromosome.

We will now present experimentations supporting the present invention.

In this study, we thoroughly assessed HERVs expression in AML and normal blood and bone marrow cells. Using a recent method to exhaustively quantify HERV retrotranscriptome in next-generation sequencing data, we show that the latter can accurately define normal and leukemic cell populations, including leukemia stem cells (LSCs) that can be characterized by a 47-HERVs signature or sub-groups thereof.

### HERV Retrotranscriptome Accurately Defines Normal Hematopoietic Cell Populations

As a first step, we examined HERVs expression in the different normal hematopoietic cell populations, assuming that distinct HERVs profiles may characterize the main cell types. Using a custom pipeline based on Telescope (20), we quantified the expression of 14,968 HERVs loci in RNA-seq data from sorted bone-marrow and peripheral blood cell populations from 9 healthy donors (n=49 samples) (19). Unsupervised hierarchical clustering based on the top 20% most variable HERVs showed a robust classification of normal hematopoietic cell types with a cluster purity of 77.6% and a corrected Rand Index of 0.61. The same approach based on genes reached a purity of 65.3% with a corrected Rand Index of 0.47.

We then sought to improve the clustering with the analysis of peaks from open chromatin regions assessed by ATAC-seq. Using the HOMER package, we applied a classic human genome annotation from gencode (v33) to annotate the set of 590,650 significant non-overlapping peaks from open chromatin regions previously defined in sorted healthy donors' bone marrow and peripheral blood cells (n=80 samples) (19). As previously described, unsupervised hierarchical clustering based on promoters elements (peaks between −100 bp and 1,000 bp away from a transcription start-site (TSS)) and intergenic elements (peaks more than 1,000 bp away from any other feature) significantly improved cluster classification, with a purity reaching 81.8%. We then re-annotated these significant peaks with a custom reference consisting of the same gencode annotation concatenated with the previously used 14,968 HERVs loci from Repeatmasker. Overall annotation showed that 16% of the total significant peaks correspond to HERVs regions. One important previously reported finding is that classification based on intergenic elements only (the so-called “distal regulatory elements”) is sufficient to classify normal hematopoietic cell populations (19). Enhanced annotation of these distal regulatory elements revealed an enrichment in HERVs, with up to 37.6% of the top 500 variable intergenic peaks corresponding to a HERV region. Plot of the total aggregated count from these regions showed a gaussian distribution surrounding HERVs' TSS, confirming the good quality of the ATAC-seq signal. Clustering of samples based on active HERVs regions (AHR, defined by peaks surrounding HERVs regions +/− 1000 or 3000 bp) further improved the clustering, reaching 88.3% cluster purity.

Altogether these results show that HERV retrotranscriptome can be used to characterize normal immature and mature hematopoietic cell populations. The improved clustering obtained with AHR defined on ATAC-seq data suggests that this retrotransciptomic signature may reflect epigenetic features associated with cell differentiation.

Acute Myeloid Leukemia Cells Show Distinct HERV Profiles Close to their Normal Cell of Origin

We next evaluated how HERV retrotranscriptome may help in distinguishing AML cells. We performed the same clustering approach, adding this time the 32 RNA-seq and 45 ATAC-seq bone marrow samples from 15 AML patients at diagnosis (19). Unsupervised clustering based on the top 20% variable AHR (+/−1,000 bp from a HERV TSS) in ATAC-seq resulted again in a good classification of normal and AML cells, with a slight increase in cluster purity compared to the top 20% most variable intergenic peaks. Clustering based on HERVs expression in RNA-seq yielded comparable results. Interestingly, leukemic blast cells (blasts) clustered with either monocytes or granulocyte-monocyte progenitor (GMP) cells, LSCs with either GMP or lymphoid-primed multipotent progenitor (LMPP) cells and pre-leukemic hematopoietic stem cells (pHSCs) with cither GMP or HSC/multipotent progenitor (MPP) cells, suggesting a clustering with their cell of origin as already described by Corces et al (19). Cluster purity based on the original cell categories do not consider these similarities and is thus a poor indicator of clustering performance in this case. Differential ATAC-count analysis centered on extended AHR (+/−20,000 bp from a HERV TSS) revealed distinct profiles between AML LSCs, blasts and pHSCs compared to their normal counterpart, with globally a chromatin more open in blasts and more closed in LSCs and pHSCs. To further characterize the role of HERVs in these AHR, we computed correlations between RNA expression of each HERV present in an AHR and its respective surrounding genes located at +/−50,000 bp. Strikingly, we found mostly positive correlations between HERVs expression and their surrounding genes. Annotation of the genes with a pre-established list of cancer-associated genes from the Conser Gene Census database (26) found several genes positively correlated with HERVs expressed in AHR. Of note, the highest correlation was found for GATA1 with ERVLB4_Xp11.23b (Pearson's R: 0.74, adjusted p-value: 8.11c-14). Using TCGA LAML RNAseq data, we then explored the association between each HERV located in an AHR and gene copy number variation (CNV) on the same cytoband. We found several HERVs correlating both positively and negatively with deletions, and mostly positively with amplifications on the same cytoband (not shown). These results show that HERVs expression profile differs according to the AML cell type and suggest that HERVs are associated with gene regulation.

### HERVs Expression as Biomarker

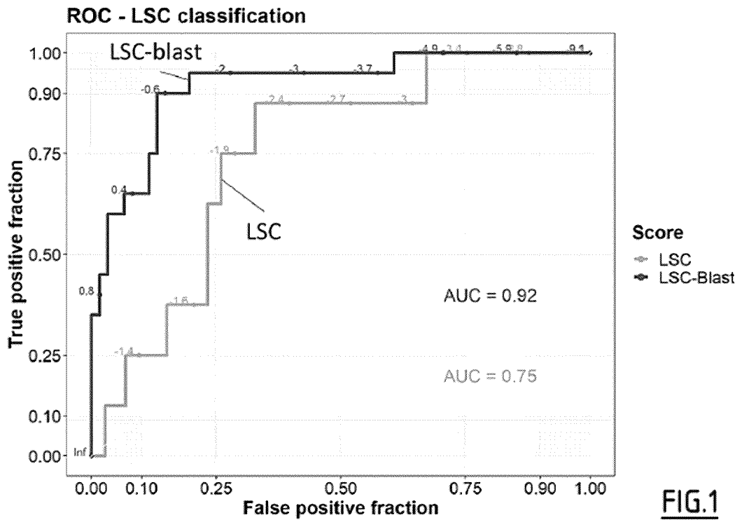

To demonstrate the value of HERVs expression as a promising biomarker, a LSC signature based on HERVs expression was established. We calculated correlations between each individual HERV and the previously published LSC17 score (18) in the 4 independent datasets. HERVs with a significant correlation with the LSC17 score (adjusted p-value <0.05) in at least 2 independent datasets were used to establish a new LSC score (see methods). A LSC signature based on 47 different HERVs was thus established (Table 1). To validate this signature, we assessed its performance in the independent dataset of sorted AML cells previously used. This signature allowed separation of LSCs versus all the other cells with an area under the curve (AUC) of 0.75 (FIG. 1). Misclassified cells were mainly attributed to the blasts group, as confirmed by the very good AUC of 0.92 when regrouping LSCs and blasts (FIG. 1). Altogether, these results show that HERVs represent biomarkers that can be used to define different AML subtypes as well as cell-specific signatures, as highlighted here for LSCs.

Using the unique information provided by the HERV retrotranscriptome, we thus built a signature based on 47 HERVs that allowed a robust classification of LSCs among normal and leukemic bone-marrow cells. Misclassified cells are mostly represented by blast cells. A LSC-HERV signature could represent an original tool to either evaluate AML prognosis (i.e. as a surrogate marker of the remaining LSCs) or minimal residual disease during follow-up.

### Prognosis Method

A bulk bone marrow sample is taken from the patient to be tested. The patient may be a patient suffering from AML, a patient being treated against AML, a patient that has been treated against AML, or patient to be diagnosed with respect to AML. The score according to the present invention is calculated using the following method.
1. Perform NGS in said sample. NGS can be either non-targeted (total or polyA RNA-seq) or targeted.
2. Quantify HERVs from NGS data
   (a) Align raw-reads to human genome reference using Bowtie2 with conservative parameters: —no-unal —score-min L, 0, 1.6-k 100 —very-sensitive-local (see 21)
   (b) Quantify HERVs using the open-source tool Telescope (see 20).
   (c) Quantify genes with any tool such as HTseq or featurecount.
   (d) Normalize expression data taking genes raw count into account.
3. Calculate the LSC score for each patient
   (a) Multiply each HERVs' normalized expression value by its coefficient provided in Table 1.
   (b) Calculate the score as the mean of each pondered HERVs' expression.

The ideal signature should be assessed with the whole 47-HERVs. In case not all HERVs are available, the core-signature has to be assessed with the sub-groups as disclosed herein, especially at least the 29-HERVs with an absolute coefficient >0.4 (see Table 1).

The more this value, the more the amount of residual LSC. Thus, the method may allow assessing a prognosis based on this value. In a first embodiment, this may be done using a relative classification, i.e. considering the continuous value for a given cohort, and classifying patients between them, patients with the lowest value having the best predicted outcome, and patient with the highest value the worse predicted outcome. In a second embodiment, this may be done using an absolute classification based on the score's terciles on the training cohort, patients with a score of more than 0.5 having a poor predicted outcome, and patients with a score of less or equal than 0.15 having a good predicted outcome.

### Methods

### Raw RNAseq Data

Raw RNA-seq data files were accessed from the NCBI Gene Expression Omnibus (GEO) portal, under the accession numbers GSE74246 for the sorted hematopoietic normal and AML cells from Corces et al. (19), GSE49642, GSE52656, GSE62190, GSE66917, GSE67039 and GSE106272 for the LEUCEGENE datasets, GSE127825 and GSE127826 for the six mTECs samples (6). TCGA LAML (22) and BEAT-AML (23) data were accessed from the NCI Genomic Data Commons (GDC) data portal (https://portal.gdc.cancer.gov/). Raw data for the AMLCG cohort (10) were directly provided by the AMLCG group.

### HERVs and Genes Expression Quantification

HERVs expression was quantified using a custom pipeline derived from Telescope (20). Briefly, RNAseq reads were aligned to a custom transcriptome using bowtie2 v2.2.1 (21) with custom parameters to keep multimaps (-k 100 —very-sensitive-local —score-min “L, 0, 1.6”). The custom transcriptome consisted in the hg38 reference transcriptome with 14,968 HERVs transcriptional units compiled from RepeatMasker annotations (20). SAM outputs were converted to BAM files using SAMtools v1.4 (33). HERVs and genes expression was then calculated using Telescope (20) and HTSeq 0.12.3 (24), respectively. Raw counts were then concatenated and normalized independently for each dataset using DESEQ2 v1.28.0 with variance stabilizing transformation (VST) (25).

### ATACseq Data

Significant peaks called from ATACseq data analysis were retrieved from the original paper (19). Briefly, peaks were called using MACS2 and filtered using a custom blacklist. A final set of 590,650 significant peaks were defined among a list of non-overlapping maximally significant 500 bp peaks ranked by their summit significance value. These significant peaks were re-annotated using HOMER with the command “annotatePeaks.pl” and two different references: Gencode v33 only and Gencode v33 with the previously used HERVs annotation. Regions containing significant peak around +/−1,000 or 3,000 bp of a HERV TSS were considered as active HER Vs regions.

### Differential ATAC-Count Analysis

For differential ATAC-count analysis, raw ATAC-seq count were retrieved from the original paper (19). Differential expression analysis between each AML populations (LSC, pHSC and Blasts) and their normal counterpart (HSC, GMP, LMPP and monocytes) was performed using DESEQ2, with cell type as a covariate. Differentially expressed regions surrounding a HERVs TSS (+/−20,000 bp) and with a FDR <5% were retained for the final plot. The rolling mean of 1,000 sequential regions, ordered by chromosome location, was then represented.

### HERVs, Genes and Copy Number Variation Correlations

HERVs located in previously defined active HERVs regions (so-called AHR+/−20,000 bp) were selected for correlation analysis. For each HERV, a list of surrounding genes located at +/−50,000 bp of their TSS was established. Pearson's correlations were calculated between the RNA expression of each HERVs and each of its surrounding gene, independently. P-values were corrected with the FDR method. Genes were then annotated using a published list of cancer-related genes from the Cancer Gene Census (26). The same list of HERVs was then used to perform correlations with CNV from the same cytoband. TCGA LAML CNV data were retrieved from the NCI GDC portal and used as is to calculate Pearson's correlations with HERVs from the same cytoband.

### Cancer Hallmark and Immune Signatures GSVA

For each hallmark of cancer (27), a unique gene signature was established based on The Molecular Signatures Database (MSigDb) Hallmark Gene Set Collection (28). When not available in MSigDb, hallmark signatures where established from Gene Ontology (GO) signatures, as previously described (29). Signature for the immune evasion hallmark was retrieved from Hubert et al. (30). Individual enrichment score where calculated from each patient by single sample gene-set variation analysis (ssGSVA), and scaled by study. The mean score for each cluster was then calculated and shown in a radar plot.

Immune signatures were obtained from Thorsson et al. (31) and calculated by ssGSVA for each sample. Unsupervised hierarchical clustering was then performed on study-scaled ssGSVA scores in each cluster.

### HERV-LSC Signature

To establish the HERV-LSC signature, correlations between the expression of each unique HERV and the validated LSC17 score (18) were computed independently in the 4 bulk RNA-seq datasets. 47 HERVs with a significant correlation (FDR-adjusted p-value <0.05) with the LSC17 score in at least 2 datasets and with concordant results (i.e. correlated in the same direction in each dataset) were retained to build the final signature. The HERV-LSC signature was then calculated as the mean 47-HERVs expression pondered by each HERV's individual mean correlation coefficient with the LSC17 score. This signature was then validated in the sorted cells from Corces et al. (19) with a classification approach, for LSC alone or LSC and Blasts. ROC curves and AUC were drawn and calculated with the plotROC R package.

### Differential HERVs Expression Analysis

Differential expression analysis was performed using DESEQ2 (25). HERVs and genes raw counts from all normal and AML datasets were merged and integrated into the same DESEQ object, using study (i.e. batch) as a covariate in the design formula. Differential expression analysis was performed for all the 4 independent bulk AML datasets and the sorted LSC and pHSC populations against each of the 42 normal tissues. Fold change were shrunk with the apeglm method (32). Features with a fold change superior to 4 (log2FC >2) and a base mean of at least 1 normalized count per million were considered overexpressed.

### Biological Samples

Bone marrow samples were collected from AML patients at diagnosis at the Centre Hospitalier Lyon Sud in Lyon, France. Samples collection was approved from the institutional review board and ethics committee (20.01.31.72653-21/20_3) and after obtaining patients' written informed consent, in accordance with the Declaration of Helsinki. BMMCs were obtained by Ficoll density gradient centrifugation (Eurobio, FR, EU) and immediately cryoconserved in foetal bovine serum (FBS) with 10% dimethylsulfoxyde (DMSO).

### MILs Growth

BMMCs were rapidly thawed at 37° C. and put in culture in RPMI medium (Gibco, FR, EU) supplemented with 8% human AB-serum (Etablissement Français du Sang, FR, EU) and high doses (6,000 UI/mL) IL-2 (PROLEUKIN aldesleukine, Novartis Pharma, CH, EU) after a 2-hours resting. Plates were then incubated for 14 days, with medium replacement when needed.

### REFERENCES

1. Consortium I H G S. Initial sequencing and analysis of the human genome. Nature. 15 févr 2001;409 (6822): 35057062.
2. Johnson W E. Origins and evolutionary consequences of ancient endogenous retroviruses. Nat Rev Microbiol. juin 2019; 17 (6): 355-70.
3. Vargiu L, et al. Classification and characterization of human endogenous retroviruses; mosaic forms are common. Retrovirology. 22 janv 2016; 13 (1): 7.
4. Kassiotis G, Stoye J P. Immune responses to endogenous retroelements: taking the bad with the good. Nat Rev Immunol. avr 2016; 16 (4): 207-19.
5. Alcazer V, et al. Human Endogenous Retroviruses (HERVs): Shaping the Innate Immune Response in Cancers. Cancers (Basel). 6 mars 2020; 12 (3).
6. Larouche J-D, et al. Widespread and tissue-specific expression of endogenous retroelements in human somatic tissues. Genome Med. déc 2020; 12 (1): 1-16.
7. Burns K H. Transposable elements in cancer. Nat Rev Cancer. juill 2017;17 (7): 415-24.
8. Attermann A S, et al. Human endogenous retroviruses and their implication for immunotherapeutics of cancer. Ann Oncol. 01 2018; 29 (11): 2183-91.
9. De Kouchkovsky I, Abdul-Hay M. ‘Acute myeloid leukemia: a comprehensive review and 2016 update’. Blood Cancer Journal. juill 2016; 6 (7): e441-e441.
10. Herold T, et al. A 29-gene and cytogenetic score for the prediction of resistance to induction treatment in acute myeloid leukemia. Haematologica. 2018; 103 (3): 456-65.
11. Alexandrov L B, et al. Signatures of mutational processes in human cancer. Nature. 22 août 2013; 500 (7463): 415-21.
12. Smith C C, et al. Alternative tumour-specific antigens. Nat Rev Cancer. août 2019; 19 (8): 465-78.
13. Brodsky I, et al. Expression of HERV-K proviruses in human leukocytes. Blood. 1 mai 1993;81 (9): 2369-74.
14. Depil S, et al. Expression of a human endogenous retrovirus, HERV-K, in the blood cells of leukemia patients. Leukemia. févr 2002;16 (2): 254-9.
15. Tobiasson M, et al. Oncotarget. 25 avr 2017;8 (17): 28812-25.
16. Kazachenka A, et al. Genome Medicine. 23 déc 2019; 11 (1): 86.
17. Deniz Ö, et al. Endogenous retroviruses are a source of enhancers with oncogenic potential in acute myeloid leukaemia. Nat Commun. 14 2020; 11 (1): 3506.
18. Ng S W K, Mitchell A, et al. A 17-gene stemness score for rapid determination of risk in acute leukaemia. Nature. 15 2016; 540 (7633): 433-7.
19. Corces M R, et al. Nature Genetics. October 2016; 48 (10): 1193-203.
20 Bendall M L, et al. Telescope: Characterization of the retrotranscriptome by accurate estimation of transposable element expression. PLOS Comput Biol. 2019; 15 (9): e1006453.
21. Langmead, B., and Salzberg, S. L. (2012). Fast gapped-read alignment with Bowtie 2. Nat. Methods 9, 357-359.
22. The Cancer Genome Atlas Research Network (2013). Genomic and Epigenomic Landscapes of Adult De Novo Acute Myeloid Leukemia. N. Engl. J. Med. 368, 2059-2074.
23. Tyner, J. W. et al. (2018). Functional genomic landscape of acute myeloid leukaemia. Nature 562, 526.
24. Anders, S., Pyl, P. T., and Huber, W. (2015). HTSeq—a Python framework to work with high-throughput sequencing data. Bioinformatics 31, 166-169.
25. Love, M. I., Huber, W., and Anders, S. (2014). Moderated estimation of fold change and dispersion for RNA-seq data with DESeq2. Genome Biol. 15, 550.
26. Sondka, Z et al. (2018). The COSMIC Cancer Gene Census: describing genetic dysfunction across all human cancers. Nat. Rev. Cancer 18, 696-705.
27. Hanahan, D., and Weinberg, R. A. (2011). Hallmarks of Cancer: The Next Generation. Cell 144, 646-674.
28. Liberzon, et al. (2015). The Molecular Signatures Database Hallmark Gene Set Collection. Cell Syst. 1, 417-425.
29. Loeffler-Wirth, et al. (2019). A modular transcriptome map of mature B cell lymphomas. Genome Med. 11, 27.
30. Hubert, M., et al. (2020). IFN-III is selectively produced by cDC1 and predicts good clinical outcome in breast cancer. Sci. Immunol. 5.
31. Thorsson, V. et al. (2018). The Immune Landscape of Cancer. Immunity 48, 812-830.e14.
32. Zhu, A., Ibrahim, J. G., and Love, M. I. (2019). Bioinformatics 35, 2084-2092.
33. Li, H. et al., and 1000 Genome Project Data Processing Subgroup (2009). The Sequence Alignment/Map format and SAMtools. Bioinformatics 25, 2078-2079.
34. Consortium, T. U. (2019). UniProt: a worldwide hub of protein knowledge. Nucleic Acids Res. 47, D506-D515.

1-11. (canceled)

12. A method of determining an HERV-LSC signature or score, preferably to prognose or classify a patient with AML or in risk of developing AML or having an AML relapse, comprising determining from a patient's sample, expression of at least 10, at least 15, at least 20, at least 25, or the 29 of the 29-HERVs N° 1-15, and 34-47 of Table 1, wherein the score is calculated as follows: one multiply each HERVs' expression value by its coefficient provided in Table 1, giving a pondered HERVs' expression for each one of those HERVs, then the score is the mean of each one of the pondered HERVs' expressions.

13. The method according to claim 12, wherein the method comprises the determination of the 29 of the 29-HERVs N° 1-15, and 34-47 of Table 1.

14. The method according to claim 12, wherein the score is assessed with additional HERVs selected from those of N° 16-33 in Table 1.

15. The method according to claim 13, wherein the score is assessed with the whole 47-HERVs N° 1-47 in Table 1, or with a subset of 30, 31, 32, 33, 34, 35, 36, 37, 38, 39, 40, 41, 42, 43, 44, 45 or 46, preferably comprising the 29-HERVs N° 1-15, and 34-47 in Table 1.

16. The method according to claim 12, the method comprising performing RNA-Seq in a sample of a patient, method in which RNA from the sample is fragmented and the fragments are reverse transcribed into cDNA fragments, or the RNA is reverse transcribed to cDNA and then fragmented to get cDNA fragments.

17. The method according to claim 16, wherein step a. comprises performing generation sequencing (NGS) in a sample of a patient.

18. The method according to claim 16, wherein the sample of the patient is a bulk bone marrow sample.

19. The method according to claim 16, wherein said cDNA fragments are sequenced and aligned back to a pre-sequenced reference human genome or human genome reference, using a sequence aligner, these alignments are tested for overlap with said HERVs' sequences, and the number of overlap reads mapped to a gene is registered for each HERVs' sequence giving its expression value.

20. The method according to claim 12, wherein the score or signature for one patient is compared to reference values or known patient groups.

21. The method according to claim 20, wherein the reference group is a group of patients of known prognosis, and their pondered HERVs expression value and their score or signature as reference has been calculated.

22. The method according to claim 20, wherein the reference is the patient itself subjected to prognosis, wherein the reference patient is before AML treatment and allows to follow treatment effect, or the reference patient is the same at the time or at the end of AML treatment and prognosis is to evaluate response to AML treatment, to evaluate minimal or residual disease during follow-up, or to determine relapse in said AML patient.

23. The method according to claim 12, wherein prognosis for a patient is determined by considering the continuous value for a given cohort, and classifying patients between them, patients with the lowest value having the best predicted outcome, and patient with the highest value the worse predicted outcome.

24. The method according to claim 12, wherein prognosis for a patient is determined by using an absolute classification based on the score's terciles on a training cohort, patients with a score of more than 0.5 having a poor predicted outcome, and patients with a score of less or equal than 0.15 having a good predicted outcome.

25. A method of treating AML in a patient, comprising the steps of:
1. prognosing or classifying the patient with AML or in risk of developing AML or having an AML relapse by determining an HERV-LSC signature or score according to the method according to claim 12, and
2. treating said patient with a cancer therapy.

26. The method according to claim 25, wherein the patient is treated with an aggressive cancer therapy when the patient has been identified as being in high risk group for AML or in risk of developing AML or having an AML relapse.

27. The method according to claim 26, wherein the aggressive therapy is an intensified chemotherapy or monoclonal antibody therapy, or an alternative therapy through enrollment into a clinical trial for a novel therapy.

28. The method according to claim 27, wherein the monoclonal antibody therapy is selected from talacotuzumab, lintuzumab, BI 835858, daratumumab, isatuximab, multivalent antibody therapy including blinatumomab, AMG 330 and antibody-drug conjugates including gemtuzumab ozogamicin, vadastuximab talirine (SGN33A) and IMGN779.

29. The method according to claim 25, wherein the patient is treated with a less aggressive therapy when the patient has been identified as being in low risk group.

30. The method according to claim 29, wherein the less aggressive therapy is standard chemotherapy.

31. The method according to claim 30, wherein the standard chemotherapy is cytosine arabinoside (Ara-c) in conjunction with an anthracycline, such as daunorubicin or the nucleoside analogue fludarabine.
